(19) United States
(12) Patent Application Publication
(10) Pub. No.: US 2012/0309706 A1
(43) Pub. Date: Dec. 6, 2012

(54) COMBINED TREATMENT OF PANCREATIC CANCER WITH GEMCITABINE AND MASITINIB

(75) Inventors: Alain Moussy, Paris (FR); Jean-Pierre Kinet, Aix En Provence (FR)

(73) Assignee: AB SCIENCE, PARIS (FR)

(21) Appl. No.: 13/576,363

(22) PCT Filed: Feb. 1, 2011
(86) PCT No.: PCT/EP2011/051348

§ 371 Date: Jul. 31, 2012

Related U.S. Application Data
(60) Provisional application No. 61/300,178, filed on Feb. 1, 2010.

(51) Int. Cl. A61K 31/7068; A61P 35/00

(52) U.S. Cl. 514/49

(57) ABSTRACT

The present invention relates to the combined treatment of pancreatic cancers, especially in patients with metastasis and in patients whose cancer is developing resistance to first line treatment with gemcitabine, comprising administration of masitinib and gemcitabine, both in appropriate dosage regimens allowing resensitisation of cancer cells to gemcitabine.

The present invention relates to the combined treatment of pancreatic cancers, especially in patients with metastasis and in patients whose cancer is developing resistance to first line treatment with gemcitabine, comprising administration of masitinib and gemcitabine, both in appropriate dosage regimens allowing resensitisation of cancer cells to gemcitabine.

### BACKGROUND OF THE INVENTION

Pancreatic cancer is a life-threatening condition. In most cases, early stages of the disease are asymptomatic and less than 20% of pancreatic cancers are amenable to surgery. Moreover, invasive and metastatic pancreatic cancers respond poorly to existing treatments in chemotherapy and radiotherapy. Overall, the National Cancer Institute (NCI) estimate that survival rate for cancer of the exocrine pancreas is less than 4% and the median survival time after diagnosis is less than a year.

The pancreas contains exocrine cells (involved in the production of pancreatic “juice”, which in turn contain enzymes important for food digestion) and endocrine cells (that produce hormones such as insulin). Both exocrine and endocrine cells can form tumours, but those formed by the exocrine pancreas are far more common. Tumours of the exocrine pancreas are likely to be cancer. Nearly all of these tumours are adenocarcinomas. Tumours of the endocrine pancreas are far less common. They are known as islet cell tumours and are divided into several sub-types. Most of these are benign, but a few are cancerous. Ampullary cancer is a special type of cancer that grows where the bile duct and the pancreatic duct empty into the small intestine. Because this type of cancer often causes jaundice, it is usually found at an earlier stage than most other pancreatic cancers.

Early diagnosis of pancreatic cancer is difficult because symptoms vary and are non-specific. Symptoms are primarily caused by mass effect rather than disruption of exocrine or endocrine functions and depend on the size and location of the tumour, as well as the presence of metastases. Common symptoms include pain in the upper abdomen (that typically radiates to the back and is relieved by leaning forward), loss of appetite and significant weight loss and painless jaundice related to bile duct obstruction. All these symptoms can have multiple other causes. Therefore, pancreatic cancer is more frequently diagnosed at an advanced stage.

According to the American Cancer Society, the lifetime risk of developing pancreatic cancer is about 1 in 79 (1.27%). The causes of pancreatic cancer are still not well understood, but several risk factors have been identified. Some of these risk factors affect the DNA of pancreatic cells, which can result in abnormal cell growth and may cause tumours to form. Briefly the main risk factors include: age, gender, ethnicity, cigarette smoking, diet, obesity and physical inactivity, diabetes, chronic pancreatitis, occupational exposures, stomach problems and family history.

Worldwide incidence of pancreatic cancer has increased markedly over the past several decades. In the United States, according to the American Cancer Society, an estimated 34,290 Americans (17,500 men and 16,790 women) will die of pancreatic cancer in 2008, making this type of cancer the fourth leading cause of cancer death overall. In Europe, estimations by the Globocan 2002, IARC show that mortality rates (11.9 per 100,000) are similar to incidence rates (11.2 per 100,000). Approximately, 95% of pancreatic cancers are adenocarcinomas, with a median survival after diagnosis of 3 to 6 months and 6 to 11 months for patients with metastatic and locally advanced disease, respectively, and an overall 5-year survival rate below 5%. Metastases, high levels of carbohydrate antigen 19-9 (CA 19-9), and an Eastern Cooperative Oncology Group (ECOG) status≧2 are all associated with a poor prognosis.

Treatment of pancreatic cancer depends on the stage of the cancer. When the disease is confined to the pancreas and clearly separated from surrounding blood vessels (i.e. local and resectable), the treatment of choice is surgery with post-operative chemotherapy and/or radiation. When the disease encases or compresses surrounding blood vessels or has extended into adjacent structure (i.e., locally advanced and unresectable), chemotherapy and/or radiation is proposed. In rare cases, when the patient responds well to treatment, the tumour may subsequently be surgically resected. When the disease has spread to distant organs (i.e., metastatic), chemotherapy is proposed. In most cases, these treatments do not represent a cure and the median survival ranges from 3 to 18 months depending on the stage of the disease. Each of these standard treatments is described in more detail below.

Surgical resection offers the only chance for a cure for pancreatic cancer. Approximately 20% of patients present with pancreatic cancer amenable to local surgical resection, with operative mortality rates of approximately 1 to 16%. Following surgery, median survival time is 14 months.

For pancreatic cancer, the benefit of radiotherapy alone is unclear and radiotherapy is mostly used in conjunction with chemotherapy (referred to as chemoradiation).

Chemotherapy may be used in patients with advanced unresectable cancer (locally advanced or metastatic) and in patients with localized disease after surgery or, sometime, beforehand in order to shrink the tumour. Gemcitabine, and to a lesser extent 5-fluorouracil (5-FU), are the chemotherapy drugs of choice to treat pancreatic cancer. Meta-analyses show that chemotherapy has significant survival benefits over best supportive care. Moreover, gemcitabine is more effective than 5-FU and gemcitabine-based combinations are more efficient than gemcitabine alone. Standard gemcitabine therapy for patients with locally advanced, unresectable, or metastatic pancreatic adenocarcinoma, provides a median overall survival (OS) of 6 months and 1-year survival rate of 21%.

The anti-metabolite gemcitabine (CAS number 95058-81-4; (4-amino-1-[3,3-difluoro-4-hydroxy-5-(hydroxymethyl) tetrahydrofuran-2-yl]-1H-pyrimidin-2-one) has the following formula:

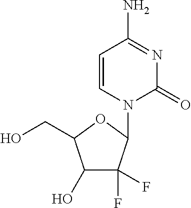

Gemcitabine replaces cytidine during DNA replication resulting in apoptosis in cancer cells. It is used in various carcinomas: non-small cell lung cancer, pancreatic cancer, and breast cancer and is being investigated for use in other cancers.

As differences between pancreatic cancer cells and normal cells are uncovered, newer drugs under development try to exploit these differences by attacking only specific targets. The hope is that these therapies will affect cancer cells while largely not affecting normal cells. Described below are some of the more advanced novel combination therapies.

Many types of cancer cells, including pancreatic cancer cells, express growth factor receptors. Among them, the epidermal growth factor receptor (EGFR) is the target of several drugs under development, including erlotinib (Tarceva) and cetuximab (Erbitux). Erlotinib in combination with gemcitabine was recently approved by EMEA for the treatment of pancreatic cancer. This combination was found to modestly extend survival in a clinical trial, with a median OS (6.24 months) 2 weeks longer than for gemcitabine monotherapy (5.91 months), a Hazard-Ratio of 0.82 (p=0.038) and 1-year survival rate of 23% (c.f. 17% for gemcitabine monotherapy treatment arm p=0.023).

Anti-angiogenesis drugs may be able to block the growth of blood vessels and thereby starve the tumour. Several are being studied in clinical trials and may be used in patients with pancreatic cancer, such as bevacizumab (Avastin), which is already used in several other types of cancer and may have some benefit against pancreatic cancer when combined with gemcitabine.

Several pancreatic cancer vaccines are under investigation. Using some abnormal aspect of pancreatic cancer cells, these vaccines should induce the immune system to recognize and kill these cells. This might cause tumours to shrink or help prevent them from reoccurring. Another form of immune therapy involves injecting patients with monoclonal antibodies targeted to cancer-specific molecules (such as the carcinoembryonic antigen). Such antibodies can be coupled to toxins or radioactive atoms and deliver them directly to the tumour cells.

A number of clinical trials are currently underway to explore the combination of gemcitabine with either cytotoxic and/or biological targeted compounds. So far, results have been disappointing, showing no or little benefit compared to gemcitabine monotherapy.

Thus, treatment of metastatic pancreatic cancer continues to be a major challenge. Despite the introduction of gemcitabine and attempts at developing combination chemotherapy regimens, pancreatic cancer remains a chemoresistant tumour. In addition, there are numerous side-effects associated with gemcitabine including myelosuppression.

The continuing poor prognosis and lack of effective treatments for pancreatic cancer highlight an unmet medical need to develop less toxic and more efficient treatment strategies that improve the clinical management and prognosis of patients afflicted with pancreatic cancer.

Masitinib is a small molecule selectively inhibiting specific tyrosine kinases such as c-kit, PDGFR, Lyn, and to a lesser extent the fibroblast growth factor receptor 3 (FGFR3) tyrosine kinase activities, without inhibiting kinases of known toxicities (Dubreuil et al, 2009, Masitinib (AB1010), a potent and selective tyrosine kinase inhibitor targeting kit; PLoS One, 4(9):e7258). The chemical name is 4-(4-methylpiperazin-1-ylmethyl)-N-[4-methyl-3-(4-pyridin-3ylthiazol-2-ylamino)phenyl]benzamide—CAS number 790299-79-5:

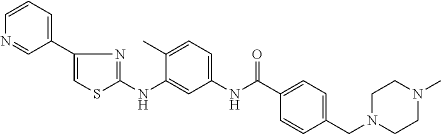

Masitinib was first described in U.S. Pat. No. 7,423,055 and EP1525200B1. A detailed procedure for the synthesis of masitinib mesilate is given in WO2008/098949.

Recently, we discovered that masitinib is able to block the FAK pathway in cells through the inhibition of FAK phosphorylation activity, without blocking its enzymatic activity and we unexpectedly discovered that the combination of masitinib mesilate and gemcitabine resulted in a down-regulation of the Wnt/β-catenin signalling pathway.

We then performed in vitro tests and we show here that gemcitabine-resistant pancreatic tumour cell lines were resensitised to gemcitabine when used in combination with masitinib, possibly in part through inhibition of the FAK pathway and/or Wnt/β-catenin signalling pathway. Preliminary in vitro data show that masitinib (1 μM) reduces FAK activity by 21% and that masitinib partially inhibits FAK auto-activation. Altogether, this could provide a mechanism of action for masitinib on pancreatic cancer through the reduction of tumour progression or the inhibition of mast cell migration and activation, or both.

The Wnt/beta-catenin signalling pathway regulates cell proliferation, differentiation and stem cell renewal (Murtaugh L C, 2008, The what, where, when and how of Wnt/beta-catenin signaling in pancreas development. Organogenesis 4: 81-86). This pathway is involved in pancreatic development and re-activation of this signalling system has been implicated in pancreatic carcinoma with reported nuclear localisation of the downstream effector beta-catenin. Down-regulation of genes involved in this signalling pathway by a combination of masitinib plus gemcitabine, may therefore contribute to accelerated death in pancreatic tumour cells as compared to gemcitabine monotherapy. Focal adhesion kinase (FAK) is a central regulator of the focal adhesion, influencing cell proliferation, survival, and migration. There is evidence demonstrating FAK overexpression in human cancer and it has been shown that FAK is required for tumour progression. The FAK signalling pathway regulates clinically relevant gene signatures and multiple signalling complexes associated with tumour progression and metastasis, such as Src, ERK, and p130Cas (Provenzano P. et al, 2008, Mammary epithelial-specific disruption of focal adhesion kinase retards tumor formation and metastasis in a transgenic mouse model of human breast cancer. Am J Pathol 173:1551-65).

Moreover, we found that the combination therapy of masitinib mesilate and gemcitabine at a particular administration regimen inhibits the growth of human pancreatic adenocarcinoma and thus represents a perspective for prolongation of survival compared to administration of gemcitabine alone. In clinical studies, and applying the regimen described hereafter, in particular administration of masitinib at a daily dose of at least 9.0 mg±1 mg/kg/day over a 28 day cycle, with possible dose escalation, together with gemcitabine at 1000±250 mg/m2 of patient surface area weekly for 3 weeks followed by 1 week of rest, every 28 days, we further found that this combination treatment prevented cancer cell metastasis and represents a perspective for prolongation of survival of patients with metastatic (grade IV) pancreatic adenocarcinoma compared to administration of gemcitabine alone.

In view of the very poor prognosis of pancreatic cancer, high occurrence of metastasis, and lack of significant survival afforded by the currently available therapies, a therapeutic strategy involving masitinib mesilate in combination with gemcitabine, or salts thereof, is shown herein to provide a novel and efficacious therapy. The advantageous aspect of this combination affords a lower dosage of gemcitabine such that the toxicity and other adverse side effects are reduced, improved efficacy of a given gemcitabine dose compared to administration of gemcitabine alone, and resensitisation of gemcitabine-refractory pancreatic cancer cells.

### DESCRIPTION OF THE INVENTION

The present invention relates to masitinib or a pharmaceutically acceptable salt thereof and gemcitabine or a pharmaceutically acceptable salt thereof for the combined treatment of pancreatic cancers, such as pancreatic adenocarcinoma, in human patients, wherein masitinib is to be administered daily at a dose of 6 mg/kg/day to 12 mg/kg/day and gemcitabine is to be administered at a weekly dose of 1000±250 mg/m² of patient surface area for up to seven consecutive weeks as a start (from 3 to 7 weeks), followed by a week off-treatment, followed by cycles of weekly dose of 1000±250 mg/m² for 3 weeks, every 28 days.

Thus, the invention encompasses the combined use of masitinib or a pharmaceutically acceptable salt thereof and gemcitabine or a pharmaceutically acceptable salt thereof, for the preparation of a medicament for the treatment of pancreatic cancers, such as pancreatic adenocarcinoma, in human patients, wherein masitinib is to be administered daily at a starting dose of 6 mg/kg/day to 12 mg/kg/day and gemcitabine is to be administered at a weekly dose of 1000±250 mg/m² of patient surface area for up to seven consecutive weeks as a start (from 3 to 7 weeks), followed by a week off-treatment, followed by cycles of weekly dose of 1000±250 mg/m² for 3 weeks, every 28 days. For Gemcitabine, it shall be understood that slight modification of the above dosage regimen is encompassed herein. For example, every 28 days means that one cycle is 3 weeks under treatment and 1 week off-treatment.

The invention also relates to a method of treatment of pancreatic cancers, such as pancreatic adenocarcinoma, in human patients, comprising administering masitinib, or a pharmaceutically acceptable salt thereof, daily at a starting dose of 6 mg/kg/day to 12 mg/kg/day and administering gemcitabine, or a pharmaceutically acceptable salt thereof, at a weekly dose of 1000±250 mg/m² of patient surface area for 3 weeks, every 28 days.

By pancreatic cancers, it is meant to refer to exocrine and endocrine pancreatic cancers, including but not limited to pancreatic adenocarcinoma.

Depending on species, age, individual condition, mode of administration, and the clinical picture in question, effective doses of masitinib are 6.0 to 12.0 mg/kg/day, especially 9.0 mg/kg/day per os, preferably in two daily intakes, administered to human patients. For adult human patients with pancreatic adenocarcinoma, a starting dose of masitinib of 9.0±1 mg/kg/day has been found to be the preferred embodiment according to the invention. For patients with an inadequate response after an assessment of response to therapy, dose escalation of masitinib to 15 mg/kg/day can be safely considered and patients may be treated as long as they benefit from treatment and in the absence of limiting toxicities. In case dose escalation is needed, it is best to increase daily dose of masitinib from the starting dose of 9.0±1 mg/kg/day by 1 or 2 mg/kg/day increment until 15 mg/kg/day is reached over a period which depends on clinical observations. For example, a single dose escalation of masitinib may take from 2 to 4 weeks. It is also contemplated herein to fully realize the therapeutic benefits of a patient-optimized dose of masitinib by dose increments smaller than the 1 to 2 mg/kg/day (100 mg). Also, dose adjustment is also to be considered to reduce toxicity in some cases. Finally, dose adjustment can be considered a dynamic process, with a patient undergoing numerous increases and/or decreases to optimize the balance between response and toxicity throughout treatment, both of which are likely to vary over time and duration of drug exposure.

Any dose indicated herein refers to the amount of active ingredient as such, for example masitinib, or gemcitabine, not to its salt form.

Pharmaceutically acceptable salts are pharmaceutically acceptable acid addition salts, like for example with inorganic acids, such as hydrochloric acid, sulfuric acid or a phosphoric acid, or with suitable organic carboxylic or sulfonic acids, for example aliphatic mono- or di-carboxylic acids, such as trifluoroacetic acid, acetic acid, propionic acid, glycolic acid, succinic acid, maleic acid, fumaric acid, hydroxymaleic acid, malic acid, tartaric acid, citric acid or oxalic acid, or amino acids such as arginine or lysine, aromatic carboxylic acids, such as benzoic acid, 2-phenoxy-benzoic acid, 2-acetoxy-benzoic acid, salicylic acid, 4-aminosalicylic acid, aromatic-aliphatic carboxylic acids, such as mandelic acid or cinnamic acid, heteroaromatic carboxylic acids, such as nicotinic acid or isonicotinic acid, aliphatic sulfonic acids, such as methane-, ethane- or 2-hydroxyethane-sulfonic, in particular methanesulfonic acid (or mesilate), or aromatic sulfonic acids, for example benzene-, p-toluene- or naphthalene-2-sulfonic acid.

In a preferred embodiment of the above depicted combined treatment, the active ingredient masitinib is administered in the form of masitinib mesilate; which is the orally bioavailable mesylate salt of masitinib—CAS 1048007-93-7 (MsOH); C28H30N6OS.CH3SO3H; MW 594.76:

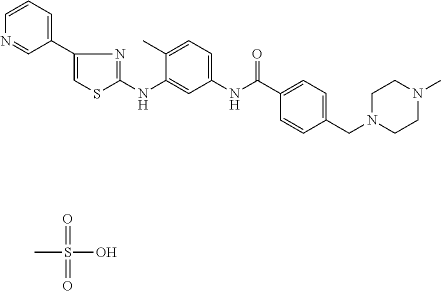

In this embodiment, the above dosage regimen does not change as the dose in mg/kg/day refers to the amount of active ingredient masitinib.

In another preferred embodiment, gemcitabine is to be administered in a cycle of 1000 mg/m² of patient surface area weekly for 3 weeks, every 28 days, which cycle is repeated as needed.

The above use or method is suited for the first line treatment of pancreatic cancers as well as for the treatment of non resectable pancreatic cancers, for resensitazing pancreatic cancer cells to gemcitabine and for blocking pancreatic cancer metastatic cells proliferation.

In the combined use or method according to the above, patients are preferably those afflicted with metastatic (grade IV) pancreatic adenocarcinoma and/or those having gemcitabine-refractory pancreatic cancer cells (gemcitabine-resistant pancreatic adenocarcinoma patient subpopulation).

Masitinib and gemcitabine may be administered in different route of administration but it is preferred to administered masitinib orally and gemcitabine by intravenous infusion or orally. Accordingly, masitinib and gemcitabine are to be administered separately, simultaneously or sequentially in time.

In still a preferred embodiment, masitinib is to be administered twice a day in the form of 100 and 200 mg tablets.

A second aspect of the invention is aimed at a kit comprising masitinib and gemcitabine, or salts thereof, together with instructions to use both masitinib and gemcitabine for the treatment of pancreatic adenocarcinoma. Advantageously, the kit comprises suitable amount of masitinib for a daily administration at a starting dose of 6 mg/kg/day to 12 mg/kg/day, preferably 9.0±1 mg/kg/day, and suitable amount of gemcitabine to be administered at a dose of 1000±250 mg/m² of patient surface area weekly for 3 weeks cycle, every 28 days, to complete at least one treatment cycle.

### KEYS TO FIGURE

FIG. 1: Tyrosine Kinase mRNA Expression Profile in Human Pancreatic Cancer Cell Lines.

(A) Messenger RNA expression of various receptor and cytoplasmic tyrosine kinases was analyzed by RT-PCR. Universal human reference total RNA was used as positive control for primers and the ubiquitous β-glucoronidase (GUS) served as an internal control for all RT-PCR reactions. (B) Tyrosine phosphorylation of proteins in response to masitinib. Mia Paca-2 cells (5×10⁶) were treated for 6 hours at 37° C. with various concentrations of masitinib. Total cell lysates were prepared and tyrosine phosphorylation was analyzed by western blot with antibodies against phosphotyrosine (anti-pTyr). Anti-GRB2 WB demonstrates comparable loading of proteins. MW=molecular weight.

FIG. 2: Masitinib Resensitisation of Resistant Pancreatic Tumour Cell Lines Mia Paca-2 and Panc-1 to Gemcitabine.

Sensitivity of pancreatic tumour cell lines to masitinib or gemcitabine as single agents, or in combination, was assessed using WST-1 proliferation assays. Four cell lines were tested for their sensitivity to masitinib (A) or gemcitabine (B). (C) Combination treatment of masitinib plus gemcitabine tested on gemcitabine resistant Mia Paca-2 cells. (D) Sensitivity of resistant Mia Paca-2 cells to various tyrosine kinase inhibitors alone (top) or in combination with gemcitabine (bottom) was analyzed in WST-1 proliferation assays.

FIG. 3: In Vivo Anti-Tumour Activity of Masitinib in a Nog-SCID Mouse Model of Human Pancreatic Cancer.

Mia Paca-2 tumour cells (10⁷) were injected into the flank of Nog-SCID mice. Treatment was initiated 28 days after tumour cell injection. The different groups were treated with either: twice weekly injections of gemcitabine (i.p. 50 mg/kg), daily oral masitinib (100 mg/kg), water (control), or combined daily oral masitinib (100 mg/kg) and twice weekly injections of gemcitabine. Mice were treated for 56 days.

FIG. 4. Kaplan-Meier Estimates of Overall Survival

(A) the ITT population; (B) according to the disease status at baseline, locally advanced vs. metastatic; and (C) performance status at baseline, KPS [70] vs. KPS [80-100].

### EXAMPLE 1

### In Vitro and In Vivo Models of Pancreatic Tumours

Preclinical studies were performed in vitro on human pancreatic tumour cell lines and then in vivo using a mouse model of human pancreatic cancer. To evaluate the therapeutic potential of masitinib mesilate in pancreatic cancer, as a single agent and in combination with gemcitabine. Molecular mechanisms were investigated via gene expression profiling.

### Methods

### Reagents:

Masitinib (AB Science, Paris, France) was prepared from powder as a 10 or 20 mM stock solution in dimethyl sulfoxide and stored at −80° C. Gemcitabine (Gemzar, Lilly France) was obtained as a powder and dissolved in sterile 0.9% NaCl solution and stored as aliquots at −80° C. Fresh dilutions were prepared for each experiment.

### Cancer Cell Lines:

Pancreatic cancer cells lines (Mia Paca-2, Panc-1, BxPC-3 and Capan-2) were obtained from Dr. Juan Iovanna (Inserm, France). Cells were maintained in RPMI (BxPC-3, Capan-2) or DMEM (Mia Paca-2, Panc-1) medium containing glutamax-1 (Lonza), supplemented with 100 U/ml penicillin/100 μg/ml streptomycin, and 10% foetal calf serum (FCS) (AbCys, Lot S02823S1800). Expression of tyrosine kinases was determined by RT-PCR using Hot Star Taq (Qiagen GmbH, Hilden, Germany) in a 2720 Thermal Cycler (Applied Biosystems).

### In Vitro Tyrosine Phosphorylation Assays:

Mia Paca-2 cells (5×106) were treated for 6 hours with increasing concentrations of masitinib in DMEM medium 0.5% serum. Cells were then placed on ice, washed in PBS, and lysed in 200 μl of ice-cold HNTG buffer (50 mM HEPES, pH 7, 50 mM NaF, 1 mM EGTA, 150 mM NaCl, 1% Triton X-100, 10% glycerol, and 1.5 mM MgCl2) in the presence of protease inhibitors (Roche Applied Science, France) and 100 μM Na3VO4. Proteins (20 μg) were resolved by SDS-PAGE 10%, followed by western blotting and immunostaining. The following primary antibodies were used: rabbit anti-phospho-GRB2 antibody (sc-255 1:1000, Santa Cruz, Calif.), and anti-phosphotyrosine antibody (4G10 1:1000, Cell Signalling Technology, Ozyme, France). These were followed by 1:10,000 horseradish peroxidase-conjugated anti-rabbit antibody (Jackson Laboratory, USA) or 1:20,000 horseradish peroxidase-conjugated anti-mouse antibody (Dako-France SAS, France). Immunoreactive bands were detected using enhanced chemiluminescent reagents (Pierce, USA).

### Proliferation Assays:

Cytotoxicity of masitinib and gemcitabine was assessed using a WST-1 proliferation/survival assay (Roche diagnostic) in growth medium containing 1% FCS. Treatment was started with the addition of the respective drug. For combination treatment (masitinib plus gemcitabine), cells were resuspended in medium (1% FCS) containing 0, 5 or 10 μM masitinib and incubated overnight before gemcitabine addition. After 72 hours WST-1 reagent was added and incubated with the cells for 4 hours before absorbance measurement at 450 nm in an EL800 Universal Microplate Reader (Bio-Tek Instruments Inc.). Media alone was used as a blank and proliferation in the absence of compounds served as positive control. Results are representative of three/four experiments. The masitinib sensitisation index is the ratio of the IC50 of gemcitabine against the IC50 of the drug combination.

### In Vivo Experiments:

Male Nog-Scid mice (7 weeks old) were obtained from internal breeding and were housed under specific pathogen-free conditions at 20±1° C. in a 12-hour light/12-hour dark cycle and ad libitum access to food and filtered water. Mia Paca-2 cells were cultured as described above. At day 0 (D0), mice were injected with 107 Mia Paca-2 cells in 200 μl PBS into the right flank. Tumours were allowed to grow for 1.5 to 4 weeks until the desired tumour size was reached (˜200 mm3). At day 28, animals were allocated into four treatment groups (n=7 to 8 per group), ensuring that each group's mean body weight and tumour volume were well matched, and treatment was initiated for a duration of 4 to 5 weeks. Treatments consisted of either: a) daily sterile water for the control group, b) an intraperitoneal (i.p.) injection of 50 mg/kg gemcitabine twice a week, c) daily gavage with 100 mg/kg masitinib, or d) combined i.p injection of 50 mg/kg gemcitabine twice a week and daily gavage with 100 mg/kg masitinib. Tumour size was measured with callipers and tumour volume was estimated using the formula: volume=(length×width2)/2. The tumour growth inhibition ratio was calculated as (100)×(median tumour volume of treated group)/(median tumour volume of control group).

### Statistical Analysis:

Relative changes in tumour volumes were compared between treatment groups using a variance analysis (ANOVA). Normality of relative changes in tumour volumes between day 28 and day 56 was first tested using the Shapiro-Wilk test of normality. In case of a positive treatment effect, treatment groups were compared two-by-two using Tukey's multiple comparison test. A p-value<0.05 was considered as significant.

### Microarray Data and Pathway Analysis:

Gene expression profiling of cell lines (from 2 μg RNA) was assessed using whole-genome Affymetrix U133 Plus 2.0 human oligonucleotide microarrays. Generation of expression matrices, data annotation, filtering and processing have been previously described [Giroux V et al., 2006 Clin Cancer Res 12: 235-241]. Microarray statistics and cluster analysis were performed by the Robust Multichip Average method in R using Bioconductor and using the Cluster and TreeView programs. Drug response signatures were generated by differential analysis, which compared the expression profile of each treated cell line with that of the untreated cell line by measuring the fold-change (treated/untreated) of each probe set. The lists of differential genes were interrogated using the Ingenuity Pathway Analysis software (Version 5.5.1-1002; Ingenuity Systems, Redwood City, Calif.) with a significance threshold for the corrected p-value<0.05. MIAME compliant array data can be accessed at (www.ebi.ac.uk/arrayexpress) using the accession number GSE17987.

### Results

### Effect of Masitinib on Pancreatic Cancer Cells In Vitro:

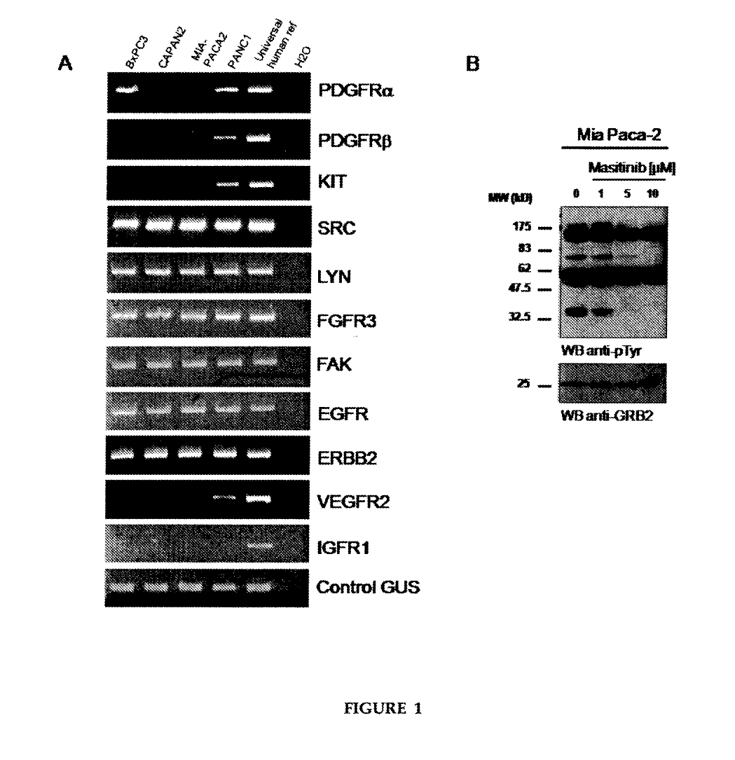

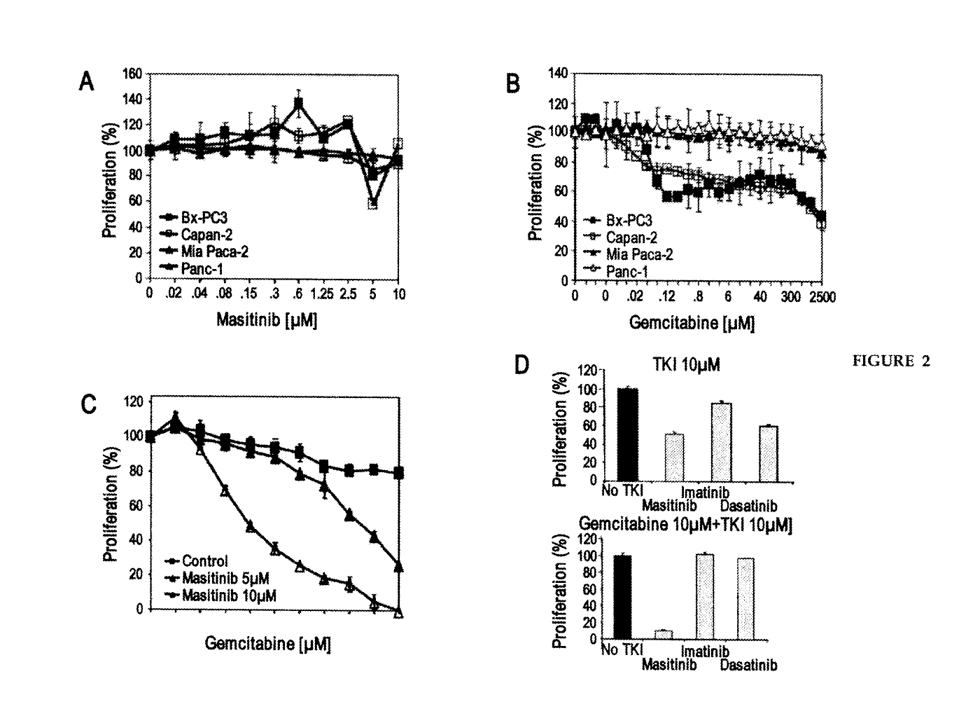

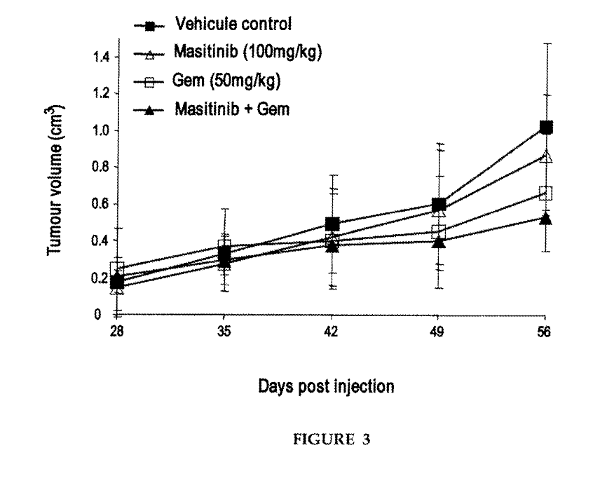

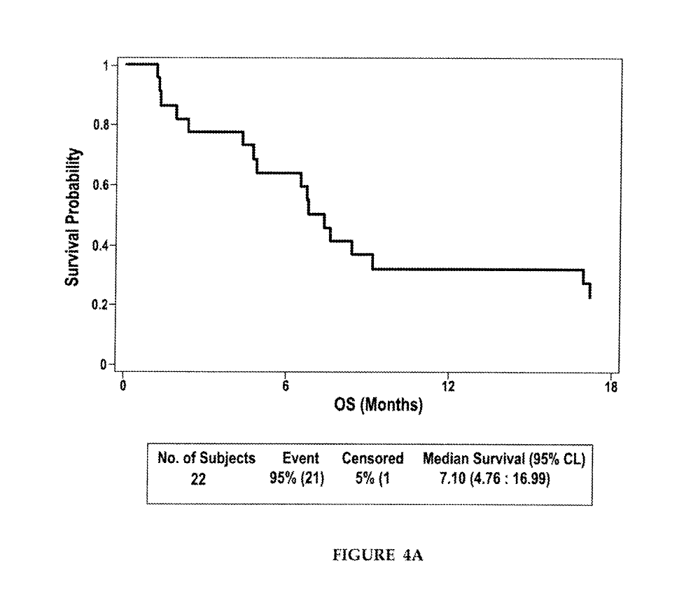

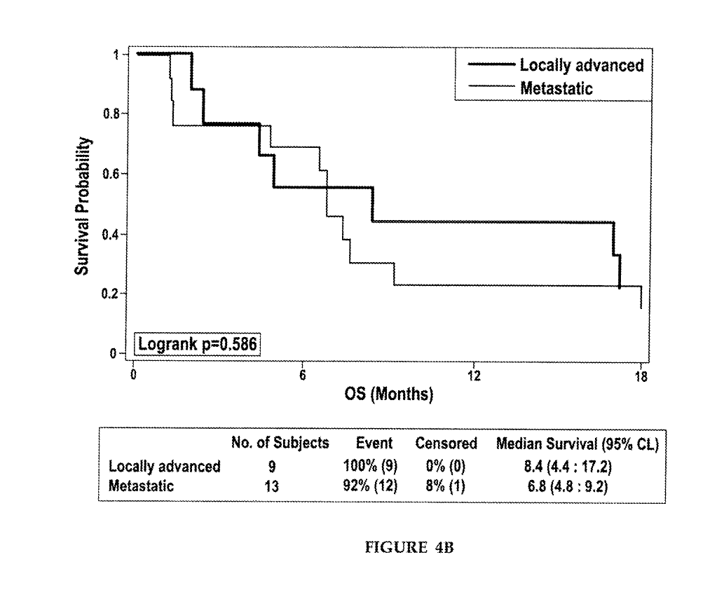

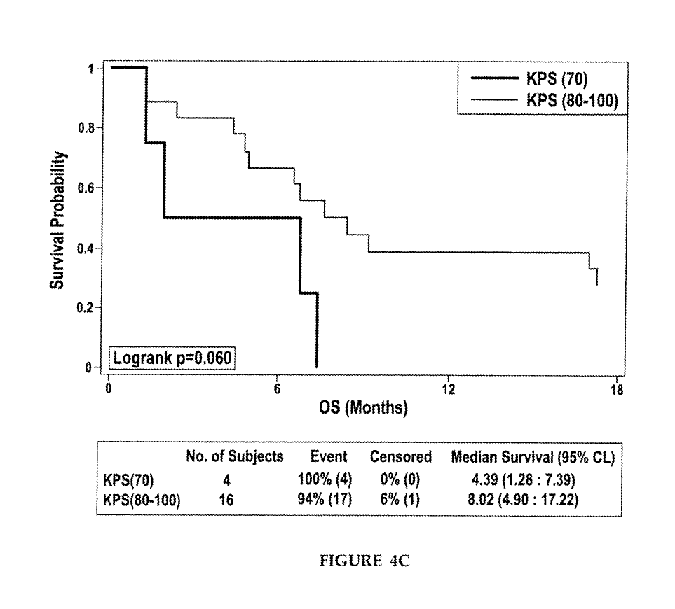

PCR with gene-specific primers was performed to determine the expression profile of masitinib's targets in the human pancreatic cancer cell lines: Mia Paca-2, Panc-1, BxPC-3 and Capan-2. C-Kit was detectable in Panc-1 cells but was undetectable in all the other cell lines. PDGFRα was expressed in BxPC-3 and Panc-1 cells while PDGFRβ was mainly expressed in Panc-1 cells. A broader profile of tyrosine kinases revealed a strong expression of the EGFR family members ErbB1 and ErbB2, src family kinases Src and Lyn, FAK and FGFR3, in all four cell lines (FIG. 1A).

To estimate the range of masitinib concentration necessary to sensitize pancreatic tumor cell lines to chemotherapy, we assessed the ability of masitinib to block protein tyrosine phosphorylation by western blot analysis in cell lysates. FIG. 1B shows a strong pattern of protein tyrosine phosphorylation at baseline in Mia Paca-2 cells. Treatment with masitinib clearly inhibited tyrosine phosphorylation at 1 μM and beyond, demonstrating that masitinib is active at these concentrations. The control protein GRB2 remained unchanged under all treatment conditions. Similar results were obtained with the other pancreatic tumour cell lines (data not shown). Based on these results, a masitinib concentration of up to 10 μM was considered appropriate to study its effect on cell proliferation.

The antiproliferative activity of masitinib or gemcitabine in monotherapy was assessed by WST-1 assays (FIGS. 2A and B). Masitinib did not significantly affect the growth of the tested cell lines, with an IC50 of 5 to 10 μM. FIG. 2B shows that gemcitabine inhibits cell lines BxPC-3 and Capan-2 with an IC50 of 2-20 μM, while Mia Paca-2 and Panc-1 cells show resistance (IC50>2.5 mM) as previously reported. Masitinib's potential to enhance gemcitabine cytotoxicity was assessed by pre-treating cell lines with masitinib overnight then exposing them to different doses of gemcitabine and recording the IC50 concentrations. Table 1 summarizes the IC50 of gemcitabine in the absence or presence of 5 and 10 μM masitinib. Mia Paca-2 cells, pre-treated with 5 and 10 μM masitinib, were significantly sensitized to gemcitabine, as evidenced by the substantial reductions (>400-fold reduction) in gemcitabine IC50 (FIG. 2C). Panc 1 cells were moderately sensitized (10-fold reduction) and no synergy was observed in the gemcitabine-sensitive cell lines Capan-2 and BxPC-3 (Table 1). These results suggest that pre-treatment with masitinib can restore cellular responsiveness to gemcitabine.

TABLE 1
IC50 concentrations (μM) of various masitinib and/or
gemcitabine treatment regimens in different pancreatic cell lines.

| | | | Gemcitabine | Gemcitabine | Sensiti- |
|---|---|---|---|---|---|
| | | Gemci- | plus 5 μM | plus 10 μM | sation |
| | Masitinib | tabine | masitinib | masitinib | Index* |
| BxPC-3 | 5-10 | 10 | 10 | 10 | 1 |
| Capan-2 | 5-10 | 2 | 2 | NA | 1 |
| Mia Paca-2 | 5-10 | >10 | 1.5 | 0.025 | 400 |
| Panc-1 | 5-10 | >10 | 8 | 1 | 10 |

*Sensitization Index is defined as the IC50 ratio of gemcitabine alone against the gemcitabine plus masitinib combination.
NA = Not available

### Results;

Comparison of masitinib to other TKIs for their potential to sensitize gemcitabine-resistant pancreatic cancer cells: Similar TKI plus gemcitabine combination experiments to those described above were performed with gemcitabine-resistant Mia Paca-2 cells to compare masitinib with imatinib (Gleevec™, STI-571; Novartis, Basel, Switzerland), a TKI targeting ABL, PDGFR, and c-Kit); and dasatinib (Sprycel, Bristol-Myers Squibb), a TKI targeting SRC, ABL, PDGFR, and c-Kit. Mia Paca-2 cell proliferation was not inhibited by imatinib alone (10 μM), whereas it was partially inhibited in the presence of low concentrations of the SRC inhibitor dasatinib (>0.1 μM); albeit with <50% of the cells remaining resistant (FIG. 2D). This suggests that Mia Paca-2 cell growth is partly dependent on SRC, which is expressed at high levels in this cell line as shown in FIG. 1A. Pre-incubation of cells with 10 μM of imatinib or dasatinib did not result in an increased response of Mia Paca-2 cells to gemcitabine as compared to masitinib (FIG. 2D). Therefore, only masitinib was able to restore sensitivity to gemcitabine in Mia Paca-2 cells.

### Effect of Masitinib on Human Pancreatic Cancer In Vivo in a Nog-SCID Mouse Model:

Preliminary experiments showed the optimal doses to use in this model (in terms of the combination's response and risk) were, masitinib at 100 mg/kg/day by gavage and gemcitabine at 50 mg/kg twice weekly by i.p. injection (data not shown). Tumours of the desired size (200 mm3) were obtained 28 days following Mia Paca-2 cell injection. The tumour size was monitored every 7 days until day 56, after which time the animals were sacrificed. FIG. 3 shows stabilization of tumour growth between day 35 and 49 in mice treated with gemcitabine or gemcitabine plus masitinib. Tumour response for each treatment group is reported in Table 2.

TABLE 2
Effect of masitinib plus gemcitabine on Mia Paca-2 pancreatic
tumours in Nog-SCID mice following 28 days of treatment.

| | | Tumour volume | | Relative change | |
|---|---|---|---|---|---|
| Treatment | Response | (mm³) | | in volume (%) | |
| group | rate | Median | Range | Mean ± SD | Range |
| Control | 0/7 (0%) | 1023 | 711-1422 | 5.4 ± 2.3 | 2.8-9.0 |
| Masitinib | 3/7 (43%) | 865 | 450-1543 | 4.8 ± 1.4 | 2.6-6.6 |
| (100 mg/kg) | | | | | |
| Gemcitabine | 6/8 (75%) | 662* | 353-1317 | 2.1 ± 1.1 | 0.7-3.6 |
| (50 mg/kg) | | | | | |
| Masitinib + | 6/8 (75%) | 526* | 166-1190 | 2.4 ± 1.8 | 0.0-5.3 |
| Gemcitabine | | | | | |

*p-value <0.05 versus control using Tukey's multiple comparison test. Responders are defined as having a smaller tumour volume than the lower range limit of the control group (i.e. 711 mm3). Relative change in tumour volume measured from day 28 to day 56.

The antitumour effect continued until day 56 (28 days of treatment) with better control of tumour growth evident in mice treated with the gemcitabine plus masitinib combination, as compared to the masitinib monotherapy or the control groups. Overall response analysis at day 56 defined a responder as having a smaller tumour volume than the lower range limit of the control group (i.e. 711 mm3). Following 28 days of treatment, 3/7 mice (43%) treated with masitinib alone were responders, with 6/8 mice (75%) responding in both the gemcitabine monotherapy and masitinib plus gemcitabine groups. Median tumour volumes were significantly reduced in the gemcitabine monotherapy and masitinib plus gemcitabine groups relative to control (p<0.05 Tukey's multiple comparison test). Although statistical significance was not demonstrated (p>0.05), the combination of masitinib plus gemcitabine appeared more potent than gemcitabine alone, with this observed trend being consistent over two separate experiments.

### Gene Expression Signature in Response to Masitinib Plus Gemcitabine Treatment:

To better understand the molecular mechanisms underlying the observed masitinib chemosensitisation, Mia PaCa-2 cells under various treatment regimens (untreated, masitinib monotherapy, gemcitabine monotherapy, or masitinib plus gemcitabine in combination), were profiled using DNA microarrays. Whole-genome clustering of the four cell samples sorted them into two opposite clusters (data not shown). The two treatment conditions with gemcitabine clustered together (left cluster), whereas cells treated with masitinib alone clustered with the untreated cells (right cluster). This result suggests that changes of gene expression in response to masitinib treatment are less numerous than those associated with gemcitabine chemotherapy, which is to be expected as masitinib is a more targeted agent. This was confirmed by the differential analysis of expression profile. Using a fold-change threshold of 2 (up-regulation) and 2 (down-regulation), we identified 971 deregulated genes after combined masitinib plus gemcitabine treatment (845 up- and 126 down-regulated); 1161 deregulated genes after gemcitabine monotherapy (1048 up- and 113 down-regulated); and only 354 deregulated genes after masitinib monotherapy (325 up- and 29 down-regulated). Results are displayed as a colour-coded matrix including all 1412 deregulated genes (data not shown). These drug response expression signatures were characterised via pathway analysis using Ingenuity software. From the 971 genes deregulated after combined masitinib plus gemcitabine treatment, 142 (100 up- and 42 down-regulated genes) were specific to this treatment, while after gemcitabine or masitinib monotherapies, 818 and 201 genes were deregulated, respectively. When considering these specific combination-regulated genes, no pathway was found as significantly over represented among the up-regulated genes. Among the down-regulated genes, one oncogenic pathway emerged as the most significantly over represented, the Wnt/β-catenin Signalling (p<0.001). The Wnt/β-catenin signalling pathway regulates cell proliferation, differentiation and stem cell renewal. This pathway is involved in pancreatic development and re-activation of this signalling system has been implicated in pancreatic carcinoma with reported nuclear localisation of the downstream effector β-catenin. Down-regulation of genes involved in this signalling pathway by a combination of masitinib plus gemcitabine, may therefore contribute to accelerated death in Mia Paca-2 cells as compared to gemcitabine monotherapy. Three other pathways altered to a lesser extent included: ERK/MAPK Signalling, CDK5 Signalling, and PI3K/AKT Signalling (p=0.016, 0.025, 0.039, respectively).

### Conclusion:

The preclinical data reported here establishes the proof that masitinib can reverse resistance to chemotherapy in pancreatic tumour cell lines. Masitinib used in combination with gemcitabine has promising potential in the treatment of pancreatic cancer, particularly in cases where the tumour has become refractory to conventional chemotherapy.

### EXAMPLE 2

### Clinical Evaluation on Patients

An open-label, multicenter, non-randomized, phase 2 clinical trial was conducted to evaluate the efficacy and safety of masitinib mesilate combined with gemcitabine in patients with advanced pancreatic cancer.

### Methods

### Patients:

Patients enrolled in this study had a histologically or cytologically confirmed non-resectable, locally advanced or metastatic pancreas adenocarcinoma with measurable tumour lesions of longest diameter≧20 mm using conventional techniques (or ≧10 mm using spiral CT scan). Patients also had to be ≧18 years old, with life expectancy≧3 months and had a Karnofsky performance status (KPS)≧70%. Exclusion criteria included inadequate organ function defined via blood test levels, history of other malignancies (except in situ carcinoma of the cervix or basal cell carcinoma of the skin) within the 5 years prior to treatment, myocardial infarction in the previous 6 months, severe/unstable angina, severe neurological or psychiatric disorders, or pregnancy. No prior or concomitant chemotherapy, radiotherapy, immunotherapy, biologic or hormonal therapy were allowed.

### Treatment:

Oral masitinib, supplied as 100 and 200 mg tablets, was administered daily at 9 mg/kg/day (corresponding to approximately 600 mg/day) divided in two intakes, during meals. Gemcitabine was administered weekly at 1,000 mg/m2 body surface area via a 30 minute i.v. infusion, for up to seven consecutive weeks, followed by a week off-treatment. Subsequent gemcitabine cycles consisted of weekly infusions for three consecutive weeks per 4-week period. Systemic corticosteroids, and/or therapeutic anticoagulation with low molecular weight heparin or a mini-dose of warfarin (e.g. 1 mg/day) were permitted. Other investigational therapies or anticancer drugs (other than gemcitabine) and certain other agents (e.g. phenyloin or high-dose warfarin) were prohibited to avoid cytochrome P450 competition. Haematopoietic growth factors were prohibited during the first 4 weeks of treatment but allowed thereafter for patients with documented cytopaenia. Patients on bisphosphonate therapy for at least 2 months prior to entry could continue this therapy.

Dose Reduction or Removal from Therapy:

If grade 3 toxicity occurred (National Cancer Institute Common Terminology Criteria for Adverse Events, NCI CTCAE v3.0 classification), treatment was suspended until resolution and then resumed at the same dosage. If grade 3 toxicity reoccurred, treatment was interrupted until toxicity resolved and then resumed with a dose reduction of 1.5 mg/kg/day for masitinib. Grade 4 toxicity required a similar interruption in treatment, but was accompanied by an immediate reduction in masitinib dosage upon resumption of therapy. Patients were withdrawn from the trial if grade 3-4 toxicities reoccurred despite dose reduction. Treatment with the other drug continued if either masitinib or gemcitabine were temporarily interrupted. Treatment was discontinued for adverse events (AEs), progression, or withdrawal of consent. Complete end of study data were collected within 2 weeks after the final treatment.

### Efficacy and Safety Assessment:

All patients who received at least one dose of masitinib were included in the Intent-To-Treat analysis (ITT population). A Data Review Committee defined the Per Protocol (PP) population of 19 patients, with three patients disqualified due to absence of any post-baseline tumour assessment. All analyses were however, performed using the ITT population unless otherwise stated. Tumour assessments were scheduled at baseline, week 4, 8, 12 and every 8 weeks thereafter. The primary efficacy endpoint was Time-To-Progression (TTP) according to the Response Evaluation Criteria in Solid Tumours (RECIST). An a priori threshold of TTP>2.3 months was defined as being a positive response for the masitinib plus gemcitabine combination. This threshold was based upon the pivotal trial for gemcitabine treatment conducted by Burris et al. [1997, J Clin Oncol 15: 2403-2413] in which an advanced pancreatic population (consisting of both locally advanced and metastatic patients) showed a medium TTP of 2.33 months. Secondary objectives were overall survival (OS), observed survival rate, best overall response (RECIST) and clinical benefit; the latter being analyzed according to methodology used in the study of gemcitabine treatment and defined as the improvement of pain intensity, analgesic consumption, PS (performance status), and weight of patients.

Analyses were performed for all patients (ITT population) and also according to subgroups based on disease status at baseline (metastatic cancer versus locally advanced tumour) or KPS status at baseline (KPS [80-100] versus KPS [70]). This exploratory subgroup analysis was conducted in part to reveal possible bias arising from inclusion of a heterogeneous patient population with differing prognoses, and to test whether any response to masitinib follow predicted prognostic trends.

TTP was defined as the delay between the first administration of treatment and disease progression. Patients who were progression-free or lost to follow-up at the time of analysis were censored at the time of their last tumour assessment for TTP. Best overall response and clinical benefit response have been previously defined [Therasse P et al., 2000 J Natl Cancer Inst 92(3):205-16; Burris H A 3rd et al. 1997 J Clin Oncol 15: 2403-2413] and were assessed every 4 weeks. OS was measured from the initiation of treatment until patient death with assessment every 4 weeks.

Safety was monitored until 17 Oct. 2008 according to the NCI CTCAE v3.0 in all patients receiving at least one dose of masitinib. Safety assessment was based upon the frequency and severity of AEs, regardless of causality.

### Statistical Analyses:

The type I (α) error was 5% (two-sided) for all analyses. A total of 20 patients were foreseen for this proof-of-concept study, to estimate a median TTP of 2 months with a precision of 1 month and an alpha value at 5%. For each modality, qualitative variables were described by their frequencies and percentage referring to filled data. The number of missing data was also specified. For comparison of qualitative variables (tumour response, clinical benefit response), Fisher exact test was used. For the TTP, Kaplan-Meier estimates were plotted and the median with its 95% confidence interval was calculated. Kaplan-Meier estimate of the TTP rates was provided at 6 and 12 months. For OS, Kaplan-Meier estimates were plotted and the median with its 95% confidence interval was calculated. As no censoring occurred until month 20, observed OS is equal to estimated OS. Survival rates were provided at 6, 12 and 18 months. The log rank test was used for comparison of survival data (OS, TTP) between subgroups according to baseline disease and performance status. All data analyses and reporting procedures used SAS v9.1 in a Windows XP operating system environment.

### Patient Disposition:

A total of 22 patients with unresectable, locally advanced or metastatic pancreatic cancer were enrolled from nine centers in France. Patient baseline characteristics are described in Table 3. The average dose of masitinib received was 8.8±0.8 mg/kg/day. The median duration of masitinib was 56 days (range 6-490) and 145 days for patients with locally advanced tumour. The median number of gemcitabine injections in the total population was eight (range 1-42), and median cumulative dose was 14,413 mg (range 1,520-47,904). One patient reported treatment-related AEs (nausea, vomiting and general physical health deterioration) that led to a reduction in masitinib dose. The main reasons for treatment termination were progression for nine patients (41%); AEs for seven patients (32%); withdrawn consent for three patients (14%); and one patient (5%) each for death; alteration of general status; and investigator's decision.

TABLE 3
Demographics and clinical characteristics of patients
Parameter ITT Population (N = 22)

| Parameter | ITT Population (N = 22) | |
|---|---|---|
| Age (years) | Median | 64 |
| | Range | 45-78 |
| Gender; N (%) | Female | 12 (55%) |
| | Male | 10 (45%) |
| Time since diagnosis (months) | Median | 0.6 |
| | Range | −0.1-6.6 |
| Median CA 19-9 (kU/mL) | Median | 0.6 |
| | Range | 0-98.8 |
| Previous surgery for pancreatic | N | 2/22 (9%) |
| cancer | | |
| Disease Status; N | KPS [80-100] | 18/22 (82%) |
| | KPS [70] | 4/22 (18%) |
| | Locally advanced | 9/22 (41%) |
| | Metastatic | 13/22 (59%) |

### Results

### Time to Progression:

Efficacy results are presented in Table 4. The primary endpoint of median TTP was 6.4 months (95% CI [2.7-11.7]). As expected, patients with locally advanced tumours had a longer median TTP than did patients with metastatic cancer (8.3 months, 95% CI [4.6-11.7] and 2.7 months, 95% CI [1.0-NR], respectively, p=0.058). Similarly, patients with a better performance status (KPS 80-100) had a longer median TTP (6.4 months, 95% CI [2.7-11.7]) than did patients with KPS [70] (0.8 month, 95% CI [0.6-1.0], p<0.0001). The estimated rates of patients without progression at 6 and 12 months were 50.8% (95% CI [NR-NR]) and 12.7% (95% CI [0.7-41.9]), respectively. All patients with KPS [70] or metastatic cancer had progressed by 6 months. For locally advanced tumour patients the estimated progression-free rates at 6 and 12 months were 68.6% (95% CI [21.3-91.2]) and 17.1% (95% CI [0.8-52.6]), respectively, and 57.0% (95% CI [NR-NR]) and 14.3% (95% CI [0.8-45.7]), respectively for patients with KPS [80-100].

### Overall Survival:

Median OS was 7.1 months (95% CI [4.8-17.0]) (Table 4, FIG. 4A). In the metastatic subgroup, median OS was 6.8 months (95% CI [4.8-9.2]) compared to 8.4 months for locally advanced patients (95% CI [4.4-17.2], p=0.59, FIG. 4B). Patients with KPS [80-100] had a median OS of 8.0 months (95% CI [4.9-17.2]), whereas it was 4.4 months for patients with KPS [70] (95% CI [1.3-7.4], p=0.06, FIG. 4C).

The survival rate of patients (ITT population) was 63.6% at 6 months (95% CI [40.3-79.9]), 31.8% at 12 months (95% CI [14.2-51.1]), and 22.7% at 18 months (95% CI [8.3-41.4]) (Table 4). For patients with KPS [80-100], survival rates were 66.7% at 6 months (95% CI [40.4-83.4]), 38.9% at 12 months (95% CI [17.5-60.0]), and 27.8% at 18 months (95% CI [10.1-48.9]); whereas patients with KPS [70] had a survival rate of 50.0% at 6 months (95% CI [5.8-84.5]), and 0.0%, at 12 months. Patients with metastatic cancer had a survival rate of 69.2% at 6 months (95% CI [37.3-87.2]) and 23.1% at 12 and 18 months (95% CI [5.6-47.5]). Patients with locally advanced disease had a survival rate of 55.6% at 6 months (95% CI [20.4-80.5]), 44.4% at 12 months (95% CI [13.6-71.9]) and 22.2% at 18 months (95% CI [3.4-51.3]).

### Best Response:

One confirmed partial response (PR) was recorded in a locally advanced cancer patient with a KPS [80-100]. In addition, four unconfirmed PR were reported. The disease control rate (partial response plus stable disease) was 72.7% (16/22, Table 4). For locally advanced patients, the disease control rate was 88.9% (8/9) and 61.5% for metastatic patients (8/13). Patients with KPS [80-100] had a disease control rate of 88.9% (16/18), whereas all patients with KPS [70] progressed immediately.

### Clinical Benefit:

Four patients had an evaluation time of less than 4 weeks and were excluded from clinical benefit analysis. Of the 18 patients evaluated, three locally advanced cancer patients (38%), with KPS [80-100] and one metastatic cancer patient with KPS [80-100], had a clinical benefit as defined previously (Table 4).

TABLE 4
Summary of efficacy outcomes with subgroup analysis according baseline status

| | | Sub analysis (disease status) | | | Sub analysis (KPS status) | | |
|---|---|---|---|---|---|---|---|
| | ITT | Locally | | | KPS [80- | | |
| | population | advanced | Metastatic | | 100] | KPS [70] | |
| | N = 22 | N = 9 | N = 13 | p-value | N = 18 | N = 4 | p-value |
| Median | 6.4 | 8.2 | 2.7 | 0.058 | 6.4 | 0.8 | <0.0001 |
| TTP | [2.7; 11.7] | [4.6; 11.7] | [1.0; NR] | | [2.7; 11.7] | [0.6; 1.0] | |
| (months) | | | | | | | |
| (95% CI) | | | | | | | |
| Patient | | | | | | | |
| without | | | | | | | |
| progression | | | | | | | |
| (%)^{a} | | | | | | | |
| 6 months | 51 | 69 | NC | | 57 | 0 | |
| 12 months | 13 | 17 | NC | | 14 | 0 | |
| Median OS | 7.1 | 8.4 | 6.8 | 0.59 | 8.0 | 4.4 | 0.06 |
| (month) | [4.8; 17.0] | [4.4; 17.2] | [4.8; 9.2] | | [4.9; 17.2] | [1.3; 7.4] | |
| [95% CI] | | | | | | | |
| Observed | | | | | | | |
| survival | | | | | | | |
| rate (%) | | | | | | | |
| 6 months | 63.6 | 55.6 | 69.2 | | 66.7 | 50.0 | |
| [95% CI] | [40.3; 79.9] | [20.4; 80.5] | [37.3; 87.2] | | [40.4; 83.4] | [5.8; 84.5] | |
| 12 months | 31.8 | 44.4 | 23.1 | | 38.9 | 0 | |
| [95% CI] | [14.2; 51.1] | [13.6; 71.9] | [5.6; 47.5] | | [17.5; 60.0] | | |
| 18 months | 22.7 | 22.2 | 23.1 | | 27.8 | 0 | |
| [95% CI] | [8.3; 41.4] | [3.4; 51.3] | [5.6; 47.5] | | [10.1; 48.9] | | |
| Disease | 72.7 | 88.9 | 61.5 | | 88.9 | 0.0 | |
| control | [49.8; 89.3] | [51.8; 99.7] | [31.6; 86.1] | | [65.3; 98.6] | [0; 60.2] | |
| rate (%) | | | | | | | |
| [95% CI] | | | | | | | |
| Clinical | N = 18 | N = 8 | N = 10 | | N = 16 | N = 2 | |
| benefit | 22.2 | 37.5 | 10.0 | | 25.0 | 0 | |
| response (%) | [6.4; 47.6] | [8.5; 75.5] | [0.3; 44.5] | | [7.3; 52.4] | [0.0; 84.2] | |
| [95% CI] | | | | | | | |

^{a}Estimated rate based upon assessable patients at relevant time-points (not the ITT population). NC: not calculable. NR: Not reached.

### Safety:

The most frequent (>10% of patients) AEs with their causalities are listed in Table 5. At the cut-off date for safety (17 Oct. 2008), all 22 patients enrolled had experienced at least one dose of masitinib. All 22 patients (100%) experienced at least one AE, of which 21 patients (95.5%) reported at least one AE suspected to be related to masitinib. One patient reported a masitinib-related grade 4 neutropenia. The most common masitinib-related, haematological grade 3 AEs were anaemia (22.7%), lymphopenia (22.7%), neutropenia (18.2%) and leucopoenia (18.2%). The most common, masitinib-related, non-haematological grade 3 AE was asthenia (13.6% of patients). A total of 506 AEs were reported, of which 261 (52%) were suspected to be masitinib-related, the majority of which were of grade 1-2 severity. One patient's death was reported to be due to several AEs (two syncopes, severe abdominal pain, hypotension, grade 2 anemia, acute renal failure and respiratory distress syndrome) and was suspected to be related to masitinib at the time of occurrence. However, masitinib had been interrupted for 6 days before these fatal AEs occurred. Since masitinib's clearance half-life is 17 hours, the complete wash-out of masitinib was probably reached. Thus, the death of this patient is most unlikely related to masitinib. Four other deaths occurred during this study but none were suspected to be treatment related.

TABLE 5
Adverse events reported in patients undergoing combination
therapy with gemcitabine and masitinib (>10% of patients).

| | | | | | | Suspected relationship to masitinib | | | | |
|---|---|---|---|---|---|---|---|---|---|---|
| | All causalities | | | | | (or not assessable) | | | | |
| PREFERRED TERM | All Grades | | Grade 3 | | Grade 4 | All grades | | Grade 3 | | Grade 4 |
| At least one toxicity | 22 | (100%) | 22 | (100%) | 4 (18.2%) | 21 | (95.5%) | 18 | (81.8%) | 1 (4.5%) |
| Haematological events | | | | | | | | | | |
| Anaemia | 15 | (68.2%) | 7 | (31.8%) | | 8 | (36.4%) | 5 | (22.7%) | |
| Neutropaenia | 10 | (45.5%) | 6 | (27.3%) | 2 (9.1%) | 6 | (27.3%) | 4 | (18.2%) | 1 (4.5%) |
| Thrombocytopaenia | 9 | (40.9%) | 1 | (4.5%) | | 6 | (27.3%) | 1 | (4.5%) | |
| Lymphopaenia | 8 | (36.4%) | 6 | (27.3%) | | 7 | (31.8%) | 5 | (22.7%) | |
| Leucopoenia | 6 | (27.3%) | 4 | (18.2%) | | 5 | (22.7%) | 4 | (18.2%) | |
| Haemoglobin | 3 | (13.6%) | 1 | (4.5%) | | | | | | |
| Non-haematological events | | | | | | | | | | |
| Nausea | 16 | (72.7%) | 1 | (4.5%) | | 14 | (63.6%) | 1 | (4.5%) | |
| Diarrhoea | 15 | (68.2%) | 2 | (9.1%) | | 11 | (50.0%) | 2 | (9.1%) | |
| Pyrexia | 13 | (59.1%) | 1 | (4.5%) | | 6 | (27.3%) | | | |
| Vomiting | 12 | (54.5%) | | | | 11 | (50.0%) | | | |
| Asthenia | 11 | (50.0%) | 5 | (22.7%) | 1 (4.5%) | 6 | (27.3%) | 3 | (13.6%) | |
| Rash | 11 | (50.0%) | 2 | (9.1%) | | 11 | (50.0%) | 2 | (9.1%) | |
| Oedema peripheral | 9 | (40.9%) | | | | 8 | (36.4%) | | | |
| Abdominal pain | 7 | (31.8%) | 1 | (4.5%) | | 4 | (18.2%) | | | |
| Constipation | 7 | (31.8%) | | | | 2 | (9.1%) | | | |
| Hypoalbuminemia | 7 | (31.8%) | 1 | (4.5%) | | | | | | |
| Pleural effusion | 7 | (31.8%) | | | | | | | | |
| Ascites | 5 | (22.7%) | | | | | | | | |
| Dyspnoea | 5 | (22.7%) | 2 | (9.1%) | | 1 | (4.5%) | 1 | (4.5%) | |
| Cough | 4 | (18.2%) | | | | | | | | |
| Mucosal inflammation | 4 | (18.2%) | | | | 1 | (4.5%) | | | |
| Abdominal pain upper | 3 | (13.6%) | | | | | | | | |
| Anorexia | 3 | (13.6%) | 1 | (4.5%) | | 2 | (9.1%) | 1 | (4.5%) | |
| Aspartate aminotransferase | 3 | (13.6%) | 2 | (9.1%) | | 1 | (4.5%) | 1 | (4.5%) | |
| Back pain | 3 | (13.6%) | | | | | | | | |
| Blood alkaline phosphatase | 3 | (13.6%) | 1 | (4.5%) | | 1 | (4.5%) | | | |
| increased | | | | | | | | | | |
| Blood bilirubin increased | 3 | (13.6%) | 1 | (4.5%) | 1 (4.5%) | 1 | (4.5%) | | | |
| Flatulence | 3 | (13.6%) | | | | 1 | (4.5%) | | | |
| General physical health | 3 | (13.6%) | 1 | (4.5%) | | 1 | (4.5%) | | | |
| deterioration | | | | | | | | | | |

### CONCLUSION

This open, multicenter, non-randomized, phase 2 study evaluated the efficacy and safety of masitinib combined with gemcitabine in patients with locally advanced or metastatic pancreatic cancer. The combination of masitinib with gemcitabine resulted in a median TTP of 6.4 months, which is above our defined limit for efficacy of 2.3 months. Considering that the baseline health status of this study's population was superior to some other studies, then taking a more conservative threshold of 4.1 months, derived from a population consisting solely of locally advanced patients receiving gemcitabine treatment [Storniolo A M et al., 1999 Cancer 85:1261-8], shows an improved efficacy with masitinib is still evident. Despite the small number of patients in this study, results are promising in regards to those published for gemcitabine monotherapy or gemcitabine plus erlotinib [Moore M J et al., 2007 J Clin Oncol 25(15):1960-6; Xie D R et al., 2006 World J Gastroenterol 12(43):6973-81; Burris H A 3rd et al., 1997 J Clin Oncol 15: 2403-2413], for which the median TTP values ranged from 2.3 to 3.8 months. Similarly, this study's median OS of 7.1 months and survival rates of 64% and 32% at 6 and 12 months, respectively, compared favorably to those of gemcitabine and gemcitabine plus erlotinib (median OS of 6 and 6.2 months, respectively and 12-month survival rates of 21% and 23%, respectively).

Because of the increased survival, other treatments received by the 17 patients who exited the study (five patients died while in the study) were assessed. Information was available from 14 of these patients. Most frequent post-study treatments were the combination FOLFOX 4 or gemcitabine (six patients), capecitabine or 5-fluorouracil (five patients) or oxaliplatine (four patients). Most of these post-study treatments were administered for a short period of time, ranging from 1 to 2.6 months.

Treatments given for more than 5 months were the combination FOLFOX 4 (two patients, 7.3 and 9.5 months, respectively), taxol (one patient, 5.9 months) and gemcitabine (one patient, over 21 months). None of these post-study treatments are novel treatments; therefore, they should not have impacted survival more than what is known from published survival data after treatment with gemcitabine, suggesting that the improved overall survival of these patients can be attributed to the addition of masitinib.

More recently, phase 2 trials evaluating the addition of a monoclonal antibody (either anti-EGFR cetuximab or anti-VEGF bevacizumab) to gemcitabine combined with a platinum derivative in pancreatic cancer showed no improvement in terms of survival over the combination of gemcitabine and the platinum derivative alone. Our data presented here appear to be similar to those of the combinations of gemcitabine with either cisplatin (median OS: 9.0 months) or oxaliplatin (median OS: 7.5 months) but the addition of a platinum derivative to gemcitabine resulted in a high incidence of grade 3 peripheral sensory neuropathy or of grade 3 or 4 myelosuppression, suggesting that masitinib might have a lower incidence of severe AEs.

The cancer's stage and the patient's performance status at enrolment are prognosis factors for survival. Indeed, patients with a poor health status at enrolment (KPS [70], 4/22 patients, 18%) survived less than a year. When these patients were excluded from the analysis, the overall survival rate at 18 months for KPS [80-100] patients was 28%. The healthiest patients, with locally advanced tumour, had very similar median OS and median TTP, which is counter-intuitive. This might be explained by the fact that four out of nine of these patients were censored for TTP because of death without progression. The delay between progression and death for the five other patients were 2.2, 8.0, 8.7, 10.8 and 11.5 months. Although the stage of cancer is usually a prognosis factor for survival, patients with metastatic cancer or locally advanced tumours had equivalent survival rates at 18 months (23% and 22%, respectively). Their median OS were not statistically different, whereas their median TTP were 2.7 months and 8.3 months, respectively. This suggests that the addition of masitinib to gemcitabine acts on the general survival of patients with metastases with a higher efficacy than on tumour progression. One hypothesis is that the partial inhibition of FAK pathway by masitinib would eliminate the most aggressive clones without inhibiting general cell proliferation, and/or prevent engraftment of new metastases. Similarly, the important overall disease control rate (72.7%) could also be explained by a possible mechanism of resensitisation of gemcitabine-resistant pancreatic tumour cells through the inhibition of FAK pathway by masitinib, as observed in our pre-clinical studies, thereby impeding adherence properties, cell migration and metastasis. It is also possible that masitinib inhibition of PDGFR could reduce the interstitial pressure within the tumour, thus increasing chemotherapy uptake [Pietras K et al., 2001 Cancer Res 61(7):2929-34; Pietras K et al., 2002 Cancer Res 62(19):5476-84]. Furthermore, masitinib may decrease tumour cells' invasiveness and tumour progression through its inhibition of c-kit by blocking mast cell migration, activation, and production of angiogenic factors including VEGF and metalloproteases [Theoharides T C, 2008 N Engl J Med 358(17): 1860-1]. Finally, the improvement of general status and pain observed in some patients could also be related to such mast cell inhibition.

This study provides promising results regarding disease-related symptom improvement and survival in advanced pancreatic cancer following gemcitabine and masitinib combination treatment.

### REFERENCES

Burris H A 3rd, Moore M J, Andersen J, Green M R, Rothenberg M L, Modiano M R, Cripps M C, Portenoy R K, Storniolo A M, Tarassoff P, Nelson R, Dorr F A, Stephens C D, Von Hoff D D (1997) Improvements in survival and clinical benefit with gemcitabine as first-line therapy for patients with advanced pancreas cancer: a randomized trial. J Clin Oncol 15: 2403-2413.
Dubreuil P, Letard S, Ciufolini M, Gros L, Humbert M, Castéran N, Borge L, Hajem B, Lermet A, Sippl W, Voisset E, Arock M, Auclair C, Leventhal P S, Mansfield C D, Moussy A, Hermine O (2009) Masitinib (AB1010), a potent and selective tyrosine kinase inhibitor targeting KIT. PLoSONE 4(9): e7258. doi:10.1371/journal.pone.0007258.
Giroux V, Malicet C, Barthet M, Gironella M, Archange C, Dagorn J C, Vasseur S, Iovanna J L (2006) p8 is a new target of gemcitabine in pancreatic cancer cells. Clin Cancer Res 12: 235-241.
Moore M J, Goldstein D, Hamm J, Figer A, Hecht J R, Gallinger S, Au H J, Murawa P, Walde D, Wolff R A, Campos D, Lim R, Ding K, Clark G, Voskoglou-Nomikos T, Ptasynski M, Parulekar W (2007) Erlotinib plus gemcitabine compared with gemcitabine alone in patients with advanced pancreatic cancer: a phase III trial of the National Cancer Institute of Canada Clinical Trials Group. J Clin Oncol 25(15):1960-6. doi: JCO.2006.07.9525[pii]10.1200/JCO.2006.07.9525
Murtaugh L C (2008) The what, where, when and how of Wnt/beta-catenin signaling in pancreas development. Organogenesis 4: 81-86.
Pietras K, Rubin K, Sjoblom T, Buchdunger E, Sjoquist M, Heldin C H, Ostman A (2002) Inhibition of PDGF receptor signaling in tumor stroma enhances antitumor effect of chemotherapy. Cancer Res 62(19):5476-84.
Pietras K, Ostman A, Sjoquist M, Buchdunger E, Reed R K, Heldin C H, Rubin K (2001) Inhibition of platelet-derived growth factor receptors reduces interstitial hypertension and increases transcapillary transport in tumors. Cancer Res 61(7):2929-34.
Provenzano P P, Inman D R, Eliceiri K W, Beggs H E, Keely P J. Mammary epithelial-specific disruption of focal adhesion kinase retards tumor formation and metastasis in a transgenic mouse model of human breast cancer. Am J Pathol 2008; 173:1551-65.
Storniolo A M, Enas N H, Brown C A, Voi M, Rothenberg M L, Schilsky R (1999) An investigational new drug treatment program for patients with gemcitabine: results for over 3000 patients with pancreatic carcinoma. Cancer 85:1261-8.
Theoharides T C (2008) Mast cells and pancreatic cancer. N Engl J Med 358(17): p. 1860-1.
Therasse P, Arbuck S G, Eisenhauer E A, Wanders J, Kaplan R S, Rubinstein L, Verweij J, Van Glabbeke M, van Oosterom A T, Christian M C, Gwyther S G (2000) New guidelines to evaluate the response to treatment in solid tumors. European Organization for Research and Treatment of Cancer, National Cancer Institute of the United States, National Cancer Institute of Canada. J Natl Cancer Inst 92(3):205-16.
Xie D R, Liang H L, Wang Y, Guo S S, Yang Q (2006) Meta-analysis on inoperable pancreatic cancer: a comparison between gemcitabine-based combination therapy and gemcitabine alone. World J Gastroenterol 12(43):6973-81.

1. (canceled)

2. A method of treatment of pancreatic cancers, such as pancreatic adenocarcinoma, in human patients, comprising administering masitinib, or a pharmaceutically acceptable salt thereof, daily at a starting dose of 6 mg/kg/day to 12 mg/kg/day and administering gemcitabine, or a pharmaceutically acceptable salt thereof, at a weekly dose of 1000±250 mg/m² of patient surface area for up to seven consecutive weeks as a start, followed by a week off-treatment, followed by cycles of weekly dose of 1000±250 mg/m² for 3 weeks, every 28 days.

3. The method according to claim 2, wherein masitinib is masitinib mesilate.

4. The method according to claim 2, wherein masitinib is to be administered at a starting daily dose of 9.0±1 mg/kg/day.

5. The method according to claim 2, wherein masitinib is dose escalated to reach 15 mg/kg/day.

6. The method according to claim 2, wherein gemcitabine is to be administered in a cycle of 1000 mg/m² of patient surface area weekly for 3 weeks, every 28 days, which cycle is repeated as needed.

7. The method according to claim 2, for the first line treatment of pancreatic cancers.

8. The method according to claim 2, for the treatment of non resectable pancreatic cancers.

9. The method according to claim 2, for resensitazing pancreatic cancer cells to gemcitabine.

10. The method according to claim 2, for blocking pancreatic cancer metastatic cells proliferation.

11. The method according to claim 2, wherein patients are those afflicted with metastatic (grade IV) pancreatic adenocarcinoma.

12. The method according to claim 2, wherein patients are patients having gemcitabine-refractory pancreatic cancer cells (gemcitabine-resistant pancreatic adenocarcinoma patient subpopulation).

13. The method according to claim 2, wherein masitinib is administered orally and gemcitabine is administered by intravenous infusion.

14. The method according to claim 2, wherein masitinib and gemcitabine, or salts thereof, are both administered orally.

15. The method according to claim 2, wherein masitinib and gemcitabine are to be administered separately, simultaneously or sequentially in time.

16. The method according to claim 2, wherein masitinib is to be administered twice a day.

17. A kit comprising masitinib and gemcitabine, or salts thereof, together with instructions to use both masitinib and gemcitabine for the treatment of pancreatic cancers, such as pancreatic adenocarcinoma.

18. A kit according to claim 17, comprising suitable amount of masitinib for a daily administration at a starting dose of 6 mg/kg/day to 12 mg/kg/day and suitable amount of gemcitabine to be administered at a dose of 1000±250 mg/m² of patient surface area weekly for 3 weeks cycle, every 28 days, to complete at least one treatment cycle.
